(19) United States
(12) Patent Application Publication
(10) Pub. No.: US 2005/0019354 A1
(43) Pub. Date: Jan. 27, 2005

(54) Topical benfotiamine treatments

(76) Inventor: Nicholas V. Perricone, Guilford, CT (US)

Correspondence Address: ST. ONGE STEWARD JOHNSTON & REENS, LLC, 986 BEDFORD STREET, STAMFORD, CT, 06905-5619, US

(21) Appl. No.: 10/625,244

(22) Filed: Jul. 23, 2003

(51) Int. Cl. A61K 31/675; A61K 6/00; A61K 7/00

(52) U.S. Cl. 424/401; 514/86

(57) ABSTRACT

Topical compositions to improve skin condition comprise an effective amount of an allithiamine, such as benfotiamine, and a carrier. Methods for improving skin condition comprise applying a composition containing benfotiamine in a dermatologically acceptable carrier to skin tissue.

### FIELD OF THE INVENTION

The present invention relates to topical compositions to improve skin condition. More specifically, the present invention relates to topical benfotiamine compositions to increase skin elasticity, reduce the appearance of fine lines, and even out skin coloring.

### BACKGROUND OF THE INVENTION

Benfotiamine is a synthetic derivative of thiamine (vitamin B1) which is more readily absorbed by the body. Benfotiamine has been traditionally taken orally and may also be intramuscularly or intravenously injected. It is a known treatment for diabetic neuropathy and reduction of nerve pain, and to generally support nerve function.

Recent studies have shown benfotiamine to block three major biochemical pathways which are destructive to endothelial cells and prevented retinopathy in diabetic rats. Nature Medicine 9: 294-9 (March 2003). In Germany, it has commonly been used to treat diabetic neuropathy, sciatica and other nerve conditions.

In treating these conditions, benfotiamine is believed to prevent sugars in cells from combining with proteins to form advanced glycation endproducts (“AGEs”). AGEs are a result of a chemical processes in which protein structure is warped by the exposure to sugars or other reactive molecules. When blood sugar levels rise in the bloodstream and in the body's cells, the cellular metabolism of sugar is overloaded and highly reactive glucose-derived intermediates called triosephosphates build-up in cells and trigger damaging biochemical reactions. In these reactions, surrounding proteins, lipids, and DNA are attacked, resulting in cell damage. Cells susceptible to high sugar levels, such as neurons and cells of the retina and kidney, will be most affected by AGEs, and as such, are prone to damage particularly in individuals afflicted with diabetes.

These damaging reactions may be prevented by channeling of triosephosphates into a safe metabolic pathway mediated by the enzyme transketolase and produding harmless metabolites rather than damaging AGEs. As thiamin (B12) is the cofactor that works with the enzyme transketolase and essentially activates it, thiamin is believed to prevent AGE formation and resulting cell damage. As benfotiamine is a highly absorbable, it effectively increases the levels of thiamin in cells and the resulting transketolase activity that shields cells from AGE damage.

While use of benfotiamine is known in the treatment of nerve conditions, its glycation-reducing effects have not been utilized in topical applications nor in treatments for skin conditions.

### SUMMARY OF THE INVENTION

Topical compositions to improve the condition of skin comprise an effective amount of an allithiamine, such as benfotiamine, and a carrier.

Methods for preventing and/or treating skin damage and/or aging comprise applying a composition containing benfotiamine in a dermatologically acceptable carrier to skin.

### DETAILED DESCRIPTION OF THE INVENTION

Glycation of proteins in the cells of the skin causes a loss of skin elasticity and the breakdown of collagen. The immediate effects of glycated proteins include inflammation, wrinkles, and brown spots or lipofuscin. Glycated proteins also produce toxic free radicals which can have multiple damaging effects on cells. The present invention recognizes this process and provides a composition and method to minimize and to treat such glycation damage.

The present invention comprises a topical benfotiamine treatment to improve skin condition by preventing glycation of proteins in cells of the skin. Benfotiamine (S-Benzoylthiamine-O-Monophosphate) is a member of the allithiamine group, a class of thiamin-derived compounds. Benfotiamine has an open-ring structure that makes it fat soluble and allows it to pass through cell membranes and be absorbed directly into cells. As such, it is the most potent of the allithiamines and makes it effective as a topical application as it can easily pass through the lipid bilayer of the cell membranes of epidermal and dermal cells.

Topical compositions containing benfotiamine according to the present invention are topically applied to and absorbed by the skin tissue. Benfotiamine activates transketolase, increasing its activity by 300%, and prevents protein glycation and AGE formation. After treatment for the recommended period of time, decreased inflammation, irritation, and erythema of the skin are observed. Elasticity and a supple feeling is returned to the skin. Fine lines and wrinkles are lightened, and skin coloring evens out. The present invention thus prevents skin aging and treats skin aging, and well as both preventing and treating skin damage including both inflammation and erythema.

Only effective amounts of topical compositions containing benfotiamine are needed to achieve the aforementioned benefits and prevent typical effects of aging on the skin. Generally, topical application to skin tissue is accomplished in association with a carrier, and particularly one in which the benfotiamine is soluble per se or is effectively solubilized (e.g., as an emulsion or microemulsion). Where employed, the carrier is inert in the sense of not bringing about a deactivation or oxidation of the benfotiamine active ingredient(s), and in the sense of not bringing about any adverse effect on the skin areas to which it is applied.

In one preferred practice of the invention, benfotiamine is applied in admixture with the dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin areas. While the carrier for the topical composition can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent(s). Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. In the preferred embodiment, the carrier is lecithin.

As noted, these ingredients can be formulated into a cream, lotion, or gel, or a solid stick, by utilization of different proportions of the ingredients and/or by inclusion of thickening agents such as gums or other forms of hydrophilic colloids. One possible embodiment is a solution used to saturate a pad used to wipe affected areas; another is a cleanser; and others are lotions, creams, and gels, which are referred to herein as dermally or dermatologically acceptable carriers, and are formulated using conventional techniques known to those of ordinary skill in the art. The term “topical composition” as used herein shall mean the complete product including the benfotiamine active ingredient, the carrier, and any adjuvants, thickeners, excipients, etc. as described herein which is applied to a person's skin.

The quantity of the benfotiamine active ingredient in the carrier may be varied or adjusted widely depending upon the particular application, the potency of the particular compound, the desired concentration. Generally, the quantity of benfotiamine active ingredient will range between 0.05% to 70% by weight of the topical composition. In one high potency embodiment, the quantity of benfotiamine active ingredient will range between 35% to 70% by weight of the topical composition. In another embodiment, the quantity of benfotiamine active ingredient will range between 20% to 35% by weight of the topical composition. In another embodiment, the quantity of benfotiamine active ingredient will range between 5% to 20% by weight of the topical composition. In a lower potency embodiment, the quantity of benfotiamine active ingredient will range between 0.05% to 5% by weight of the topical composition. Other embodiments contain from about 0.1% to about 10% by weight, more narrowly from about 0.25% to about 5% or about 0.25% to 7% by weight. Generally, lower concentrations of benfotiamine active ingredients in a carrier are suitable, depending upon the application regimen and the active and adjunct ingredients employed.

Generally in the practice of methods of the invention, the topical composition is topically applied to the skin areas, such as that of the face, at predetermined intervals often as a moisturizer, tinted foundation, cleanser, toner, lotion, cream, or gel, it generally being the case that gradual improvement is noted with each successive application. Insofar as has been determined based upon clinical studies to date, no adverse side effects are encountered. It is an advantage of the invention that compositions of the invention do not require a pharmaceutical prescription.

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition. Preservatives include, but are not limited to, C1-C3 alkyl parabens and phenoxyenthanol, typically present in an amount ranging from about 0.5% to about 2.0% by weight percent, based on the total composition. Emollients, typically present in amounts ranging from about 0.01% to 5% of the total composition include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers, typically present in amounts from about 1% to about 10% by weight of the composition, include, but are not limited to, stearic acid, cetyl alcohol, stearyl alcohol, steareth 2, steareth 20, acrylates/C10-30 alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. Antioxidants, typically present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. As mentioned above, particularly preferred antioxidants are those that provide additional benefits to the skin such as ascorbyl palmitate. (See additional ingredients and methods in U.S. Pat. Nos. 4,775,530, 5,376,361, 5,409,693, 5,545,398, 5,574,063, 5,643,586, 5,709,868, 5,879,690, 5,965,618, 5,968,618, 6,051,244, 6,162,419, and 6,191,121 to Perricone).

Buffering agents are employed in many compositions. Preferably, the amount of buffering agent is one that results in compositions having a pH ranging from about 4.5 to about 8.5, more preferably from about 5.5 to about 8.5, most preferably from about 6.5 to about 8.0. Typical buffering agents are chemically and physically stable agents commonly found in cosmetics, and can include compounds that are also adjunct ingredients such as citric acid, malic acid, and glycolic acid buffers.

Some embodiments of this invention contain at least one other adjunct ingredient in addition to benfotiamine. Adjunct ingredients include, but are not limited to one or more of: lipoic acid; α-hydroxy acids such as glycolic acid or lactic acid; ascorbic acid and its derivatives, especially fatty acid esters of ascorbic acid; or tocotrienols and tocotrienol derivatives and vitamin E compositions enriched with tocotrienols or tocotrienol derivatives.

Many embodiments employ more than one adjunct ingredient. As used herein, the term “α-hydroxy acid” has reference to and encompasses the general class of organic compounds containing at least one hydroxy group and at least one carboxyl group, and wherein at least one hydroxyl group is located on the α-carbon atom. Typically, the compounds are organic acids having at least one carboxylic acid group and at least one hydroxyl group on the α-carbon atom, and may contain other functional groups including additional hydroxyl and carboxylic acid moieties. Preferred α-hydroxy acids and/or α-hydroxy acid derivatives are less bulky structurally so that they penetrate the skin well, and thus have a backbone of from one to three carbon atoms such as those set out in U.S. Pat. No. 5,965,618 at column 6 lines 4 to 29. Where employed, glycolic and/or lactic acid or their derivatives are preferred; glycolic acid is especially efficacious.

Fat-soluble fatty acid esters of ascorbic acid (vitamin C) is employed as an adjunct ingredient in other embodiments, alone or in combination with α-hydroxy acids. The more oxidation-resistant saturated fatty acid esters of ascorbic acid are preferred, including, but not limited to, ascorbyl laurate, ascorbyl myristate, ascorbyl palmitate, ascorbyl stearate, and ascorbyl behenate. Ascorbyl palmitate is used in one embodiment.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1. A topical composition to improve skin condition, comprising: an effective amount of benfotiamine, and a carrier.

2. The topical composition of claim 1, wherein the carrier comprises lecithin.

3. The topical composition of claim 1, further comprising at least one adjunct ingredient selected from the group consisting of a lipoic acid, an α-hydroxy acid, a fatty acid ester of ascorbic acid, and mixtures of any of these.

4. The topical composition of claim 1, wherein the composition contains from about 0.05% to about 70% by weight benfotiamine.

5. The topical composition of claim 1, wherein the composition contains from about 35% to about 70% by weight benfotiamine.

6. The topical composition of claim 1, wherein the composition contains from about 20% to about 35% by weight benfotiamine.

7. The topical composition of claim 1, wherein the composition contains from about 5% to about 20% by weight benfotiamine.

8. The topical composition of claim 1, wherein the composition contains from about 0.05% to about 5% by weight benfotiamine.

9. The topical composition of claim 1, wherein the composition contains from about 0.25% to about 7% by weight benfotiamine.

10. A method for the prevention of skin damage comprising: applying a composition containing benfotiamine in a dermatologically acceptable carrier to skin tissue.

11. A method for the treatment of skin damage comprising: applying a composition containing benfotiamine in a dermatologically acceptable carrier to skin tissue.

12. A method for the prevention of skin aging comprising: applying a composition containing benfotiamine in a dermatologically acceptable carrier to skin tissue.

13. A method for the treatment of skin aging comprising: applying a composition containing benfotiamine in a dermatologically acceptable carrier to skin tissue.

14. A method in accordance with claims 10, 11, 12, or 13 wherein said composition further comprises one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.

15. A method in accordance with claims 10, 11, 12, or 13, wherein the composition contains from about 0.05% to about 70% by weight benfotiamine.

16. A method in accordance with claim 15, wherein the composition contains from about 5% to about 20% by weight benfotiamine.

17. A method in accordance with claim 15, wherein the composition contains from about 0.05% to about 5% by weight benfotiamine.

18. A method in accordance with claim 15, wherein the composition contains from about 0.25% to about 7% by weight benfotiamine.

19. A topical composition to improve skin condition, comprising: an effective amount of an allithiamine, and a carrier.

20. The topical composition of claim 19, wherein the allithiamine consists of benfotiamine.

21. A method for the prevention of skin aging comprising: applying a composition containing allithiamine in a dermatologically acceptable carrier to skin tissue.

22. A method for the treatment of skin aging comprising: applying a composition containing allithiamine in a dermatologically acceptable carrier to skin tissue.

23. A method in accordance with claims 21 or 22, wherein the allithiamine consists of benfotiamine.
